(19) United States
(12) Patent Application Publication
(10) Pub. No.: US 2003/0153032 A1
(43) Pub. Date: Aug. 14, 2003

(54) Method of treating the surface of antigen-presenting cells

(76) Inventor: Ulrich Zimmermann, Waldbrunn (DE)

Correspondence Address: Matthew P. McWilliams, Buchanan Ingersoll P.C., Eleven Penn Center, 1835 Market Street, 14th Floor, Philadelphia, PA, 19103-2985, US

(21) Appl. No.: 10/345,824

(22) Filed: Jan. 16, 2003

(30) Foreign Application Priority Data
Jan. 18, 2002 (DE) DE 10201856.1

(51) Int. Cl. C12P 21/06; C12N 5/08

(52) U.S. Cl. 435/68.1; 435/372

(57) ABSTRACT

A method is described of treating the surface of antigen-presenting cells before modification with antigens from diseased cells. In this method the antigen-presenting cells or components thereof are exposed to the effect of proteases, after which the proteases are removed.

### SUBJECT OF THE INVENTION

The present invention relates to a method of treating the surface of antigen-presenting cells before a modification by way of antigens from diseased cells.

### TECHNICAL BACKGROUND

In order to treat tumours attempts are being made to subject a body to stimulation of the immune system, whereby endogenic or exogenic dentritic cells are modified and supplied to the body. Dendritic cells are able to present antigens so that they are recognised by the T-cells. The dendritic cells act as immunoactive cells, which depending on the relevant antigens, exhibit immunostimulating properties. The formation and properties of dendritic cells are described, for example, by K. Shortman et al. in “Stem Cells” volume 15, 1997, page 409 ff. Modification of the dendritic cells with antigens means that the antigens are incorporated in processed form into the surface of the dendritic cell. As antigens, certain model peptides (see P. Paglia et al. in “Minerva Biotechnologica” volume 11, 1999, page 261 ff) or antigens formed from the relevant tumour cells are used for example. The following methods of charging the dendritic cells with antigens of tumour cells are known.

Methods of modifying the dentritic cells include, for example, electrofusion, the use of tumour cell lysates and physical interaction in long-term incubation.

As is know, the fusions of antigen-presenting cells, such as dendritic cells, are still associated with difficulties. The problems are mainly due to the fact that the cells for charging and/or fusion are still not optimally prepared.

One disadvantage consists in the surface of the antigen-presenting cells, like dendritic cells, is often difficult to access for fusion, as the fusion takes place via the lipid phase of both adhering membranes (antigens and antigen-donating cells and antigen-presenting cells).

Another difficulty lies in the use of autologous (the patient's own) antigen-presenting cells. These are not recognised as for eign by the patient's immune defence system. However, this is the precondition if an optimum immunostimulating effect is to be achieved.

Surface modifications of biological cells have already been described. From “Naturwissenschaften”, vol. 68, page 577, 1981 (U. Zimmermann and G. Pilwat) a cell fusion between egg cells and other biological cells fusion brought about in an electrical field is known, whereby either the egg cells or the other biological cells are treated with pronase before or during the electrical fusion. The results showed that with an increasing pronase concentration the cells were stabilised against the electrical field. Sukhorukov et al. (“J. Membrane Biol.”, vol. 132, page 27, 1993), report that treatment with proteases under iso-osmolar conditions strongly changes the electrical properties of the plasma membrane in the electrical field. Corresponding measurements of the membrane capacity and membrane conductivity have been carried out with cells treated with pronase.

### OBJECT OF THE INVENTION

It is therefore the object of the present invention to prepare antigen-presenting cells in such a way that they can easily be charged and/or fused with antigens. A further object of the present invention is to be seen in modifying, particularly autologous, antigen-presenting cells so that they are suitable for immunisation, i.e. that the patient's immune defence system recognises them as foreign and initiates optimum immunostimulating effects.

### SUMMARY OF THE INVENTION

These objects are met with the inventive methods in accordance with claim 1. The dependent claims relate to preferred embodiments of the method according to the invention.

The present invention discloses a method of treating the surface of antigen-presenting cells before modification with antigens from diseased cells, in which the antigen-presenting cells or components thereof are exposed to the effect of proteases and the proteases are subsequently removed.

Surprisingly it has been found that the method in accordance with the invention is an optimum preparation measure for the modification of the antigen-presenting cells. Through the weak effect of proteases on the cells, the surface proteins of the antigen-presenting cells are only slightly digested. This results in the cells being able to be brought into contact and/or fused more easily. Especially in the case of autologous antigen-presenting cells it has been found that through the pre-treatment with proteases their immunological properties are changed and the antigen-presenting cells are easily recognised as exogenic by the body.

### PREFERRED EMBODIMENTS OF THE INVENTION

The proteases that can be used in accordance with the invention can be exopeptidases, endopeptidases, proteinases or mixtures thereof. It is also possible to use mixtures of various exopeptidases or proteinases.

It has been found that proteases such as those used for the dissociation of tissues or for the subcultivation of monolayer cultures are particularly suitable.

Trypsine, dispase and/or pronase are preferably used as proteases. Trypsine is an arginine- and lysine-specific endoprotease, dispase is a metalloprotease, which is particularly suitable for sensitive cell cultures, and pronase is a mixture of proteolytic enzymes of Streptomyces griseus.

Preferred concentration ranges are 0.1 to 0.25% for trypsine, 0.025 to 0.1% for trypsine-EDTA, 0.6 to 2.4 enzyme units (U)/ml for dispase and 0.1 to 1 mg/ml for pronase. Particularly preferred are ranges of 0.1 to 0.15% for trypsine, 0.025 to 0.5% for trypsin-EDTA, 0.6 to 1.5 enzyme units (U)/ml for dispase and 0.1 to 0.5 mg/ml for pronase (1%=1g/100 ml).

It is essential that for modifying the surface proteins of antigen-presenting cells only small concentrations of the proteases are used. As a rule the protease manufacturer's instructions are observed for incubation. A guideline value is normally the lower concentration range. In some cases it is even advisable to remain below the indicated concentration range. An important criterion in selecting the concentration of the protease to be used is that it is important to prevent the proteins being too strongly or even completely digested in the membrane.

As with the protease concentration, short action times of the enzyme(s) should be observed. Here too the manufacture's details relating to the lower limit can be observed. This also applies to the temperature and the pH value of the incubation reaction.

Generally, the values of the enzyme concentration ranges, incubation temperatures and time are dependent on the cell type (cell line, culture conditions) and also on the enzyme itself (manufacturer, purity, activity).

In addition, with regard to the protease pre-treatment in accordance with the invention, it is possible to fall back on established literature concerning routine methods of general cell handling and sub-cultivation. For example, the key work of Toni Lindl, “Zell-und Gewebekultur” (4^{th} edition, Spektrum Akademischer Verlag, Heidelberg-Berlin) can be cited here.

Incubation with the protease takes place in a protein-free medium, normally in PBS solution (phosphate-buffered NaCl solution), as proteins in the medium also act as a substrate for the proteases. In practice the antigen-presenting cells are washed in protein-free solutions before being treated with proteases and are finally incorporated into these solutions.

In many cases it is advisable to interrupt the protease treatment, even if low enzyme temperatures and short action times are being used, in order to guarantee that the surface proteins of the antigen-presenting cells are not too strongly digested. The treatment can be interrupted in various ways, which include the following procedures for example:

repeated washing of the batch with enzyme-free solutions;

adding excess protein, which then competes for the enzymes with the surface proteins and finally repeated washing with enzyme-free solutions (possible with the addition of protein);

adding specific and non-specific protease inhibitors and repeated washing with enzyme-free solutions;

inhibiting the enzyme through changing the temperature, generally reducing the temperature and repeated washing with enzyme-free solutions at low temperature, and

possibly inhibiting the proteases through changing the pH value, whereby, however, possible damage to the cells must be taken into account.

With regard to specific and non-specific protease inhibitors, commercially available ones can be resorted to. For example, the company CALBIOCHEM supplies proteases as well as their known inhibitors. The appropriate proteases/inhibitor pairs can be selected from the relevant product descriptions.

Before modification of the antigen-presenting cells the proteases must be separated, as only the surface proteins of the antigen-presenting cells should be prepared for modification. The antigens of the subsequently added diseased cells and/or components obtained therefrom must not be modified. If this should occur, no specific immune response to these antigens would be possible.

Cells such as those described in German patent application 100 33 470.9 can also be used as antigen-presenting cells. These include dendritic cells, T-cells, B-cells or mastoid cell, with preference being given to dendritic cells.

The antigens of diseased cells to be transferred to the antigen-presenting cells can originate, for example, from tumour cells or tumour cell components, bacteria, virus/virus shells, stem cells, bone marrow cells or epithelial cells.

The antigen-presenting cells used in accordance with the invention are allogenic or autologous. However, the present method in accordance with the invention is particularly suitable for use with autologous cells, because with these, as stated in the introduction, the antigen-presenting cells can be prepared in such a way that they are recognised as exogenic by the body and the immune defence system is optimally stimulated.

After being treated with protease and after separation of the proteases, the antigen-presenting cells and the antigens for the modification are exposed to an electrical field. These modification processes are generally in the form of cell fusions. These fusions are carried out, for example, through contacting by means of dielectrophoresis and subsequent fusion through electroporation.

The modification is preferably carried out in a suspension. In the suspension the suspended particles are exposed to the aforementioned electrical field so that the different cell types or cell components attract each other. Contacting under the action of the electrical field is sufficient to exclusively transfer membrane components with the antigens of the diseased cells or cell components to the antigen-presenting, e.g. dendritic, cells.

In a preferred embodiment of the method according to the invention, membrane vesicles of the antigen-presenting cells (e.g. dendritic cells) are pre-treated with protease, which are then modified with intact, antigen-presenting cells, such as dendritic cells and antigens from diseased cells. Obtained in the case of such a modification are dendritic cells, for example, with both modified and unmodified surface antigens.

The present method according to the invention is excellently suited to the preparation of antigen-presenting cells before modification with antigens. Through the action of proteases the surfaces of the antigen-presenting cell are changed in such a way that these cells can easily come into contact with each other in the subsequent stage of modification. This is a particular advantage in the case of fusion

Furthermore, through protease pre-treatment in the case of autologous antigen-presenting cells, the surface is modified, i.e. its configuration changes, in such a way that these cells, when charged with an antigen, are easily recognised as exogenic by the body's immune defence system. To this extent it is guaranteed that the medically advantageous autologous antigen-present cells can be suitable used for immunisation.

1. A method for treating the surface of antigen-presenting cells before the modification with antigens from diseased cells, in which the antigen-presenting cells or components thereof are exposed to the effect of proteases and the proteases are subsequently removed.

2. The method according to claim 1, characterised in that exopeptidases, endopeptidases, proteinases or mixtures thereof are used as proteases.

3. The method according to claim 2, characterised in that a proteinase is used which belongs to a group comprising trypsine, dispase and pronase.

4. The method according to claim 1, characterised in that the protease surface treatment is interrupted.

5. The method according to claim 1, characterised in that dendritic cells, T-cells, B-cells or mastoid cells are used as antigen-presenting cells.

6. The method according to claim 5, characterised in that the cells are allogenic or autologous.

7. The method according to claim 1, characterised in that after being treated with proteases the antigen-presenting cells and antigens for a modification of the antigen-presenting cells are exposed to an electrical field.

8. The method according to claim 7, characterised in that the treated antigen-presenting cells and the antigens are combined.

9. The method according to claim 8, characterised in that a fusion by means of dielectrophoresis and electroporation is carried out.

10. The method according to claim 7, 8 or 9, characterised in that the modification is carried out in a suspension.

11. The method according to claim 10, characterised in that only membrane components with the antigens of the diseased cells or components thereof are transferred to the pretreated antigen-presenting cells.

12. The method according to claim 1, characterised in that membrane vesicles of the antigen-presenting cells are treated with protease, which are subsequently modified with intact antigen-presenting cells and antigens from diseased cells.
